(19) United States
(12) Patent Application Publication
(10) Pub. No.: US 2013/0276539 A1
(43) Pub. Date: Oct. 24, 2013

(54) METHOD FOR NON-DESTRUCTIVE CONDITION ASSESSMENT AND STRUCTURAL MONITORING OF CONCRETE RAILROAD TIES IN TRACK

(71) Applicant: RJ LEE GROUP, INC., Monroeville, PA (US)

(72) Inventors: KEITH E. WAGNER, Latrobe, PA (US); Matthew J. Perricone, Pittsburgh, PA (US); Richard J. Lee, Murrysville, PA (US); J. Michael Wilmoth, Pittsburgh, PA (US)

(21) Appl. No.: 13/866,272

(22) Filed: Apr. 19, 2013

Related U.S. Application Data
(60) Provisional application No. 61/636,124, filed on Apr. 20, 2012.

(51) Int. Cl. G01N 33/38

(52) U.S. Cl. CPC G01N 33/383
USPC 73/595; 73/584

(57) ABSTRACT

A non-destructive testing method to evaluate the structural integrity of pre-stressed concrete railroad ties in track to identify deterioration and the eventual loss of bond between the reinforcing wires and the surrounding concrete. The application of an impact echo system to the end of each railroad tie will identify ties that have deteriorated to the extent that structural cracks have formed. The method includes employing a tool having a plurality of transducers and a single impinger to impart sound waves through a top surface of the railroad tie and to receive and evaluate the echoes of the sound waves to determine if the tie is structurally sound or structurally unsound.

### 1. FIELD OF THE INVENTION

The present invention relates to methods for testing and evaluating concrete materials employing non-destructive evaluation (NDE) techniques, and interpreting data to determine structural integrity of the concrete materials. The methods of the present invention are useful, in particular, for testing and evaluating concrete railroad ties.

### 2. DESCRIPTION OF PRIOR ART

A variety of techniques historically have been employed to determine the condition of cross ties to ensure they are capable of holding the rails at a proper gauge to avoid derailments. Visual assessments are typically limited to assessing the surface condition and are complicated since most of the tie surface is covered by ballast. The ballast must be removed from all sides of the tie for a visual evaluation to be reliably performed. Further, many of the known techniques were originally designed to assess wooden ties and may not be suitable for assessing the structural integrity of concrete ties. Wooden ties exhibit marked different structural properties than their concrete counterparts.

Sonic transducers also have been employed in assessment procedures for railroad ties. There are disadvantages associated with known methods to evaluate the structural condition of both concrete and wooden railroad ties underneath the rail seat. For example, these methods may lack an ability to detect cracks that result from the deterioration/recession of reinforcing wire in the ties prior to their propagation to the rail seat where tie performance and railroad safety can be compromised.

Impact Echo (IE) is a well-established NDE technique that is widely used in the assessment of concrete structures. There are hand-held devices manufactured and commercially available which are field deployable and equipped with a transducer that can detect the echo of a sound wave that is imparted by an impinger of the same device. It is known that the echo can represent a defect or a free surface within the material. However, it is not necessarily discernible whether the echo is “real” or what the signal actually means. This data as currently handled may require interpretation by a highly experienced user. Thus, there is a need for the technology to be capable of detecting deterioration and wire recession in concrete railroad ties resulting in a reliable identification of damaged ties that can be replaced prior to actual loss of gauge.

Further, there is a need for an in-track method that can reliably identify concrete railroad ties that have structural defects that are not detectable without the time and expense of rail line closures and ballast removal. In the absence of such a method, concrete railroad ties with loss of wire bond may not be reliably identified until their ability to hold gage is impacted, they suffer a loss of mass and/or railroad safety is affected. Furthermore, to be applicable in the field for the millions of concrete ties that are in service, use of this technique must be easily applied, robust and interpretable by users which may not be highly experienced and/or may not have a scientific background.

### SUMMARY OF THE INVENTION

The present invention provides a method to assess the structural integrity of pre-stressed concrete railroad ties through detection of structural cracks present in the tie end emanating from reinforcing wire that has lost bond with the surrounding concrete.

In one aspect, the present invention provides a device for performing an in-track condition assessment of a concrete railroad tie utilizing impact echo pulses. The device includes a base, a plurality of transducers attached to the base which are capable of detecting an echo of a sound wave, and a single impinger connected to the plurality of transducers which is capable of imparting a sound wave.

In certain embodiments, three transducers are employed. Further, an indexing feature can be connected to the base and/or a mechanism can be utilized that allows the plurality of transducers to independently adjust to provide proper contact with the railroad tie.

Further, the device can include an assessment mechanism to compare a nominal thickness of the railroad tie with an apparent depth measurement to determine if the railroad tie is structurally sound or structurally unsound. The assessment mechanism can be capable to detect at least one condition selected from deterioration, cracking and wire recession capable of leading to structure failure. The assessment mechanism can further indicate a re-check of the railroad tie at a time interval. Wherein the apparent depth is equal to or less than the nominal thickness but greater than a distance from a top surface of the railroad tie to a bottom wire line, the railroad tie is determined to be structurally sound.

In another aspect, the present invention provides a system for performing an in-track condition assessment of a plurality of concrete railroad ties utilizing impact echo impulses. The system includes a track mechanism and a plurality of devices as above-described, which are mounted to the track mechanism. The track mechanism moves in a linear direction to position each of the plurality of devices on an upper surface of an end of the railroad tie.

In still another aspect, the present invention provides a method for performing an in-track condition assessment of a concrete railroad tie utilizing impact echo pulses. The method includes positioning a tool, or device as above-described, on a top surface toward an end of the railroad tie, imparting a sound wave from the impinger below the top surface of the railroad tie, detecting with the transducers an echo of the sound wave, determining a depth measurement from the echo, comparing the depth measurement to a nominal thickness of the railroad tie and assessing if the railroad tie is structurally sound or unsound.

it is an object of the present invention to provide an efficient and economical method to identify concrete ties in track with structural deficiencies caused by deterioration and the loss of wire bond in real time.

It is a further object of the present invention that the method employed can be used by a trained (but not necessarily highly experienced) operator and interpreted by the same in real time.

It is a further object of the present invention to provide a scalable method capable of being expanded to reduce time per tie with manual assessment or automated assessment to evaluate hundreds of railroad ties per hour.

It is a further object of the present invention to provide a scalable method that can be combined with other NDE techniques (e.g., cameras, ground penetrating radar, GPS, rail cant measurements, RFID tags, and the like) to provide a cohesive assessment of tie, track, and bed conditions.

It is another object of the present invention to establish a scientific basis to identify ties with structural defects across manufacturers, tie designs, geographic locations, and concrete age.

It is another object of the present invention to provide efficient and scientifically accurate concrete tie structural assessments in real time resulting in risk reduction and improvement to railroad safety with significant cost advantages.

These and other objects of the present invention will be more fully understood from the following description of the invention with reference to the drawings appended hereto.

### BRIEF DESCRIPTION OF THE DRAWINGS

FIG. 1A is a front view of a conventional concrete railroad tie and the typical level of ballast.

FIG. 1B is a side view of the concrete railroad tie shown in FIG. 1A.

FIG. 1C is a cross-sectional view of the concrete railroad tie shown in FIG. 1A.

FIGS. 2A and 2B are a bottom view and side view, respectively, of a conventional IE device.

FIG. 3A illustrates the application of the IE device in FIGS. 2A and 2B to a railroad tie (shown in cross-section) and the cone of detection (or sensing volume) produced therefrom.

FIG. 3B illustrates the application of the IE device in FIGS. 2A and 2B to a railroad tie (shown in cross-section) and the limited ability of the cone of detection shown in FIG. 3A.

FIG. 4A is a bottom view of an IE device in accordance with certain embodiments of the invention.

FIG. 4B is a front view of an IE device and its application to a railroad tie (shown in cross-section), in accordance with certain embodiments of the invention.

FIG. 4C is a front view of an IE device with an indexing device and its application to a railroad tie (shown in cross-section), in accordance with certain embodiments of the invention.

FIG. 5 is a front view of a system including a plurality of IE devices simultaneously testing a plurality of railroad ties (shown in cross-section), in accordance with certain embodiments of the invention.

FIG. 6 is a visual depiction of the scientific basis for structurally sound and structurally unsound ratings based on the apparent depth of the tie, in accordance with certain embodiments of the invention.

### DETAILED DESCRIPTION OF THE PREFERRED EMBODIMENTS

As employed herein, the term “IE device” means any tool that has the ability of detecting and interpreting the echo of a sound wave imparted into a material, either by an impinger as a part of a device or external to that device.

As employed herein, the term “apparent depth” means the depth value reported by the IE device when analyzing the signal from the imparted sound wave during testing as determined by appropriate calibration or computation.

As employed herein, the term “rating” means the comparison between the apparent depth and the known dimensions of the reinforcing wire pattern and will have the value of structurally sound or structurally unsound. Further, the rating may indicate that a re-check of the tie is recommended at a particular interval, e.g., follow-up.

As employed herein, the term “TIECHECK™” refers to the multi-transducer IE device and interpretation (structurally sound/structurally unsound/re-check) of the reported apparent depth.

Pre-stressed concrete railroad ties are known to exhibit multiple types of failure modes. The type and location of cracking often identifies the cause of deterioration which can indicate responsibility for repair and/or replacement of the ties. For example, it is common industry practice for the railroad company to accept responsibility to repair and/or replace ties that exhibit cracking between the rails (center binding) because the cracks in this location are typically induced by loading caused by poor bed conditions which is the responsibility of the railroad company.

Other deterioration or cracking modes such as cracks beneath the rail seat typically indicate that a tie is in a state of imminent failure and cannot reliably or safely provide the intended performance. Ties that exhibit cracking at the top surface of the tie ends may or may not be structurally compromised. However, experience has shown that cracks that occur between reinforcing wires or extend from wire ends to the side of the tie are generally a source of structural concern and are indicative of loss of wire bond and thus structural integrity. The location of these cracks is commonly below the ballast line and is difficult, if not impossible, to visually assess. The present invention is capable of detecting and assessing this condition. Reliable identification of ties in this condition will allow one, e.g., the railroad company, to make timely arrangements for tie replacement prior to catastrophic failure of the tie or loss of performance that may impact railroad safety.

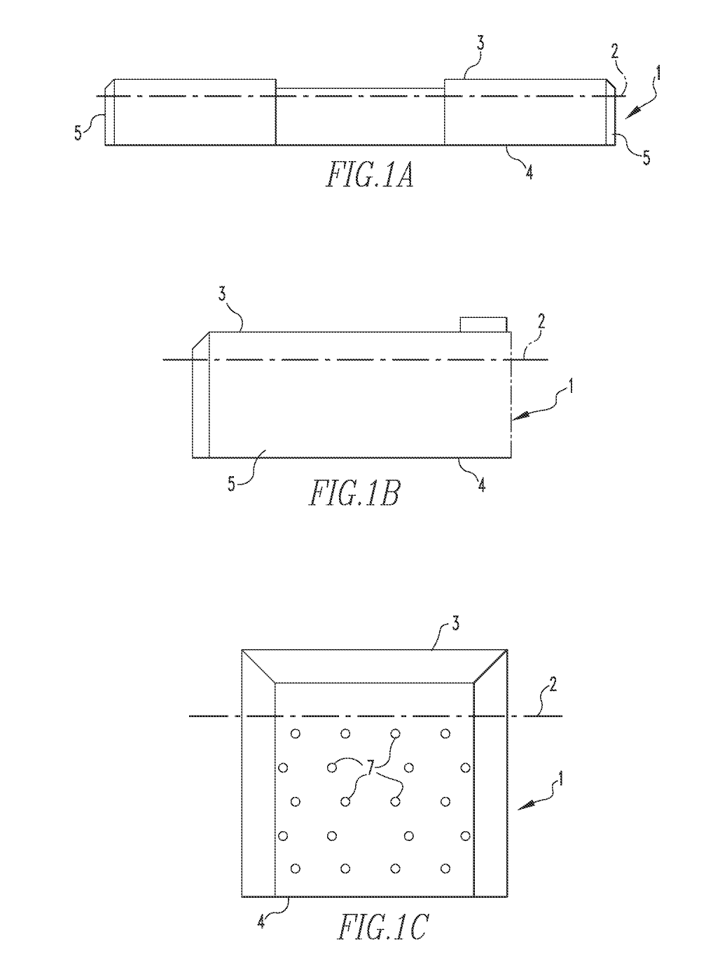

FIGS. 1A and 1B show a front view and a side view, respectively, of a typical concrete railroad tie 1 and level of ballast 2. The railroad tie 1 includes a top surface 3, a bottom surface 4 and end surfaces 5. FIG. 1C shows a cross-sectional view of the concrete railroad tie 1 including the ballast 2 and steel reinforcing rebar 7.

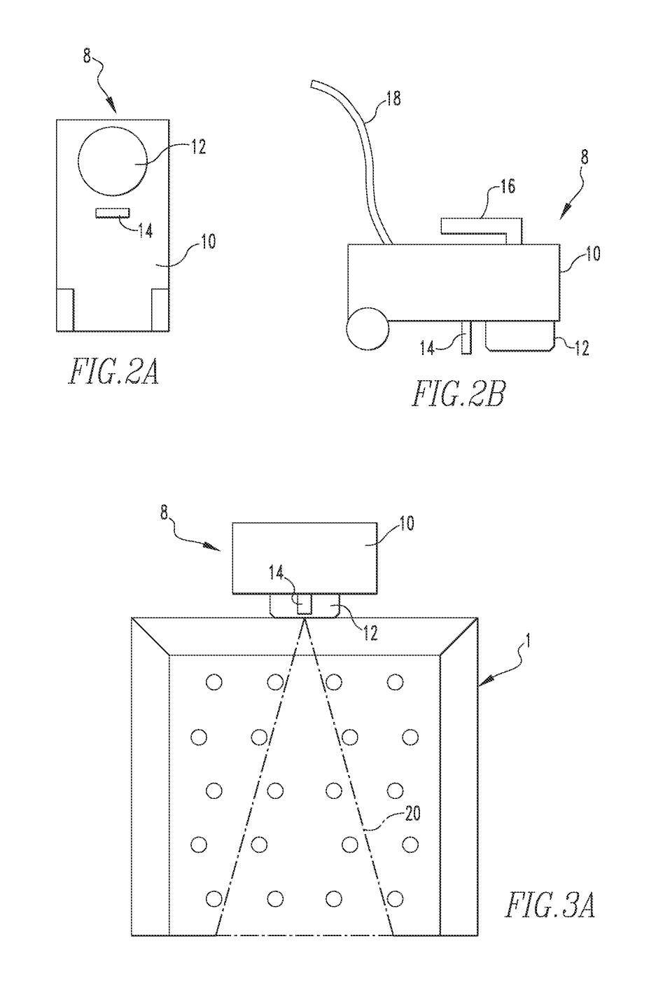

IE devices for use in assessing concrete materials have been in existence for a significant amount of time, and the technology is field deployable and well suited to manual application. A conventional IE device utilizes impact echo pulses and includes a sensor head, e.g., a transducer, which is placed in contact on a top surface of an end of a concrete railroad tie, and is operable to detect sound wave(s), echo(s) of sound wave(s), generated/imparted by an impinger which is located adjacent to or near the sensor head. FIGS. 2A and 29 show a bottom view and a side view, respectively, of a conventional IE device 8. FIG. 2A shows a base 10 and, a transducer 12 and an impinger 14 which are mechanically and electrically mounted to the base 10. The base 10 can include a container, e.g., box, that holds the electronics associated with the transducer 12 and impinger 14. When the impinger 14 is not in operation, it is typically recessed within the base 10. FIG. 2B further shows a handle 16 for gripping the device and a connector 18 for transmitting the data to a storage and/or recording mechanism (not shown).

In an embodiment, the core IE technology employed is the NDE360 product that is manufactured and distributed by Olson Instruments.

Conventional IE technology has the capability to provide information on the apparent thickness of the concrete railroad tie and is sensitive to internal cracking, particularly along the top reinforcing wire line. In order to provide this information, the IE device must be positioned at the top surface of a tie end. However, the transducer has a limited cone of collection. As a result, defects and/or cracks that fall outside of the cone of collection may not be detected.

FIG. 3A shows a cross-sectional view of the railroad tie 1, and a front view of the base 10, the transducer 12 and the impinger 14 (as shown in FIGS. 2A and 2B). Further, FIG. 3A shows a cone of collection 20 generated by the transducer which represents the limited collection ability of the transducer 12 and impinger 14. That is, any cracks or defects that are located outside of the cone of collection 20 are not identified by the IE device 8.

FIG. 3B shows the cross-sectional view of the railroad tie 1, and the front view of the base 10, the transducer 12 and the impinger 14 as shown in FIG. 3A, and furthermore shows the limited ability of the cone of collection 20 to identify cracks 22 which are located outside of the cone of collection 20. The failure of the IE device 8 to detect defects or cracks in the railroad tie is particularly evident at the top portion 24 (or top row of wires 26) where the cone of collection 20 (e.g., the area of collection) is the narrowest.

It is contemplated that the collection area produced by a single transducer is not acceptable to determine that a railroad tie is structurally sound since the area encompassed by the cone is limited to only a portion of the end of the tie. There may not be any defects or cracks detected in the cone of collection, however, as previously discussed, the area outside of the cone may, in fact, contain defects and cracks (that are undetected) and may result in the railroad tie being structurally unsound. Thus, a determination that a railroad tie is structurally sound based on data obtained from one transducer may be misleading. It is therefore contemplated that more than one measurement needs to be taken in order to cover the area of the tie end which is outside of the cone of collection associated with the transducer. It is contemplated to move the IE device, e.g., single transducer, along a distance on the end of the top surface of the railroad tie multiple times to obtain multiple readings. It is estimated that three readings would be necessary to encompass the area of the tie end for most conventional tie designs. However, this process of taking multiple readings, i.e., three readings, can be cumbersome in practice when there are a significant number of railroad ties that require assessment and evaluation.

Thus, there is a need to scale up this method using a multiplicity of transducers without having to employ a multiple IE devices. In accordance with the present invention, a single IE device with multiple transducers and a common impinger addresses this need. The multiple transducers and single, common impinger can be selected from conventional equipment and components known in the art.

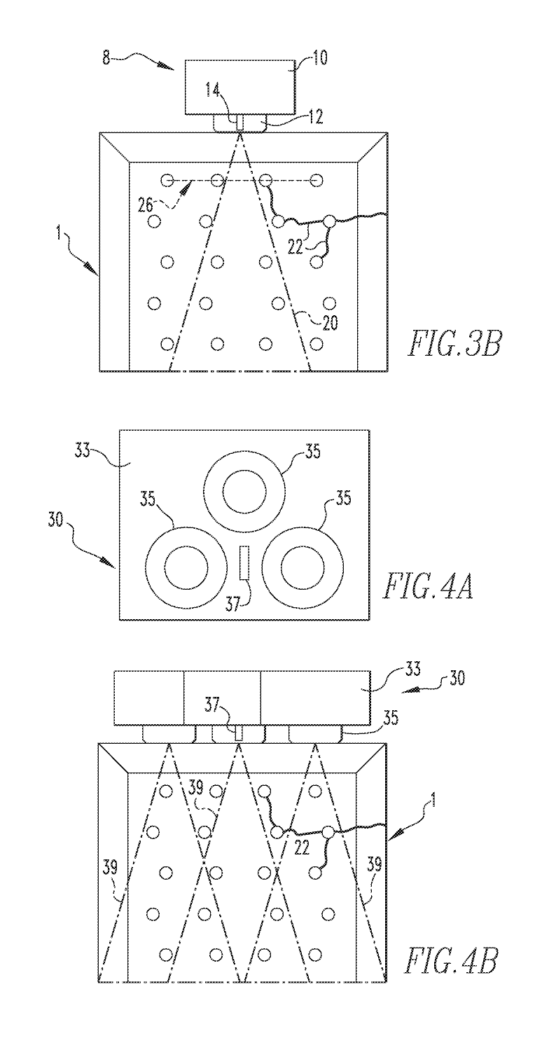

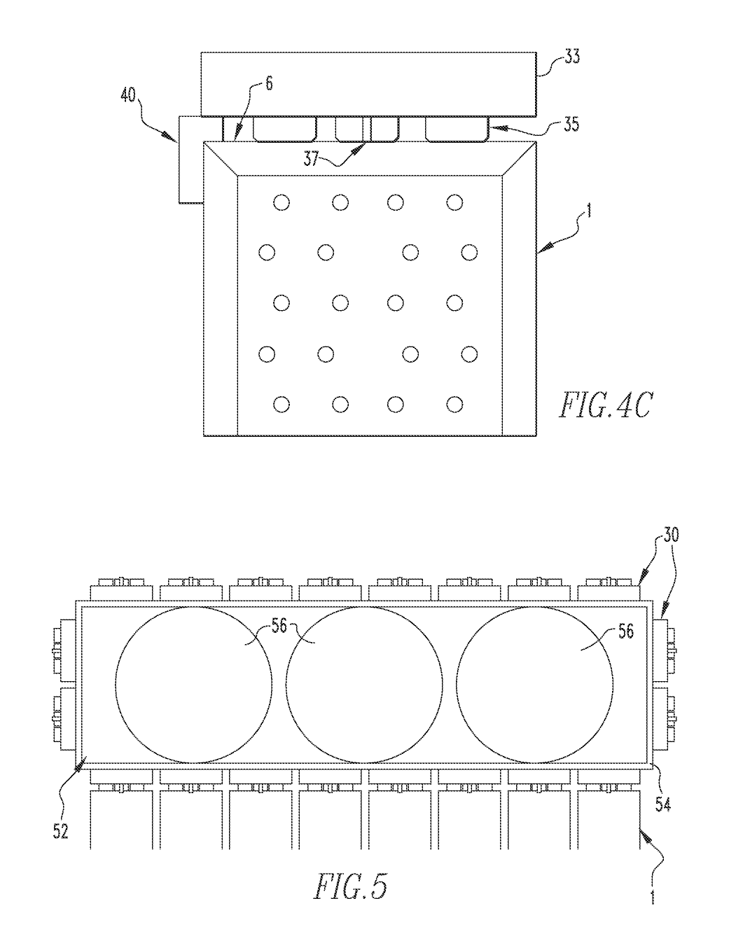

FIG. 4A shows a bottom view of a multiple transducer device 30 in accordance with certain embodiments of the present invention which includes a single base 33 with three transducers 35 and a single impinger 37 each positioned on the base 33. Modifications of the sensor (e.g., transducer) geometry to adjust for multiplicities of tie design may vary. Thus, the transducers 35 can be arranged on the base 33 in varying configurations. Further, more than three transducers 35 may be used depending on the particular design and dimensions of the railroad tie.

FIG. 4B shows a front view of the multiple transducer device 30 shown in FIG. 4A including the base 33, the three transducers 35 and the single impinger 37. Further, FIG. 4B shows that the cones of collection 39 generated by the three transducers 35 are effective to substantially cover the end area of the railroad tie 1. Furthermore, FIG. 4B shows that the cracks 22 that were undetectable by the cone of collection 20 in FIG. 3B are detectable by the multiple cones of collection 39.

FIG. 4C shows the multiple transducer device 30 (e.g., referred to as TIECHECK™ device) as shown in FIG. 4B wherein, in a preferred embodiment, an indexing feature 40 is connected to the base 33 to ensure consistent placement on the top surface 6 of the railroad tie 1. In certain embodiments, the indexing feature 40 can include physical positioning attachment(s), software, and or photographic image recognition (all not shown).

Moreover, in certain embodiments, the transducers 35 can independently adjust to maintain proper surface contact with the railroad tie 1. This feature, as well as the indexing feature 40 and the positioning attachment (not shown) are optional and not required.

In an automated embodiment of this invention, the TIECHECK™ technology is part of a multi-device automated mobile unit that can simultaneously assess in-track tie condition, rail condition, and track bed condition. This automation can maximize the number of ties analyzed per unit time. FIG. 5 shows a plurality of the multiple transducer device 30 as shown in FIGS. 4A, 4B and 4C. Further, FIG. 5 includes a mechanism 52 that is employed to provide linear movement of the plurality of devices 30. In FIG. 5, the plurality of devices 30 are mounted on a track 54 which is rotatable around wheels 56 to move and position the plurality of devices 30 on the plurality of railroad ties 1. In certain embodiments, simultaneous and synchronized deployment of the multiple TIECHECK™ devices on an automated mobile unit may be employed by a rotating tank tread arrangement. Another embodiment can include vehicle-mounted rotating wheels with multiple TIECHECK™ devices. Yet another embodiment can include articulated arrays of TIECHECK™ devices that simultaneously deployed on multiple ties and then are lifted and repositioned ahead of the array (in front of it) in a leap-frog type motion.

As used herein, TIECHECK™ device refers to the multi-transducer IE device described therein and, an assessment mechanism and/or rating system that classifies/differentiates structurally sound ties from those that will have an abbreviated service life. The present invention allows trained but not necessarily highly experienced operators to interpret the results and identify structurally unsound ties in real time.

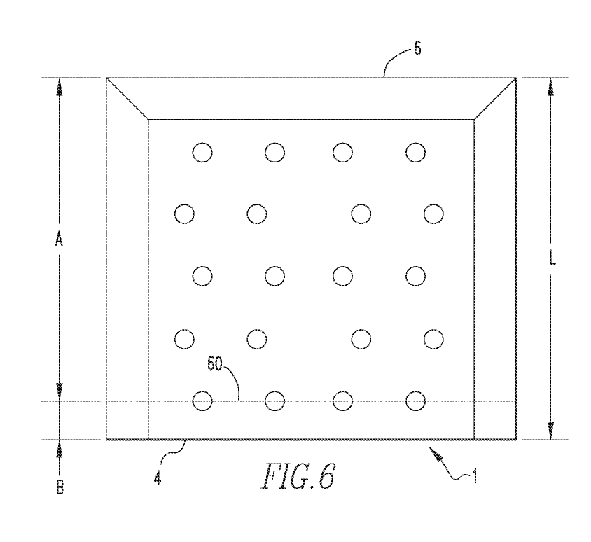

This rating system is based on comparison of the nominal thickness of the tie end and the apparent thickness as measured by the impact echo technique. FIG. 6 shows a cross-section of the railroad tie 1 having a plurality of reinforcing rebar 7. The nominal thickness of the tie 1 is identified by the letter L. Concrete ties that are structurally sound will have an apparent depth equal to or less than L, but greater than the distance A from the top of the tie to the bottom wire line 60. The letter B identifies the distance from the bottom wire line 60 to the bottom surface 4 of the tie 1. Thus, TIECHECK™ system ratings are as follows:

A≦Apparent Depth≦L=Structurally Sound

Apparent Depth<A=Structurally Unsound

Apparent Depth>L=Structurally Unsound

In certain embodiments, the ratings can include an indication for follow-up, e.g., a re-check of the railroad tie at a particular interval. For instance, the rating can include a determination of structurally sound but identify that a re-check of the tie is recommended at a time period in the future.

The TIECHECK™ technology provides a robust system and method for ratings of ties from multiple manufacturers provided that the nominal thickness, L, and the distance to the bottom row of wires, A, are known.

In certain embodiments, the invention employs handheld IE technology capable of providing a depth measurement from which the strongest echo emanates from a defect within the concrete railroad tie. The basis for tie rating is the time of detection of the return pulse (depth of the echo), which can be compared to the wire pattern in the railroad tie. When detecting how sound waves travel through a material, the speed at which that occurs plays a crucial role in interpretation. A structurally sound tie has a depth that reflects the actual height of the tie because the echo is from the bottom of the tie. However, a tie may have smaller depth than its height due to a crack which opens an internal free surface or a tie may have a longer depth than its height due to several smaller defects which slow down the sound wave travel.

The methods and systems of the invention are operable to assess structural integrity of railroad ties of varying designs and dimensions that are known in the art, and can assess deterioration and wire recession of the ties.

While specific embodiments of the invention have been described in detail, it will be appreciated by those skilled in the art that various modifications and alternatives to those details could be developed in light of the overall concept of the disclosure. Accordingly, the particular embodiments disclosed are meant to be illustrative only and not limiting as to the scope of the invention which is to be given the full breadth of the appended claims and any and all equivalents thereof.

What is claimed is:

1. A device for performing an in-track condition assessment of a concrete railroad tie utilizing impact echo pulses, comprising:
a base;
a plurality of transducers attached to the base which are capable of detecting an echo of a sound wave; and
a single impinger connected to the plurality of transducers which is capable of imparting a sound wave.

2. The device of claim 1 wherein the plurality of transducers is three transducers.

3. The device of claim 1 wherein the base comprises a container which holds associated electronics for the plurality of transducers and the single impinger.

4. The device of claim 1 further comprising an indexing feature connected to the base.

5. The device of claim 1 further comprising a mechanism that allows the plurality of transducers to independently adjust to provide proper contact with the railroad tie.

6. The device of claim 1 further comprising an assessment mechanism to compare a nominal thickness of the railroad tie with an apparent depth measurement to determine if the railroad tie is structurally sound or structurally unsound.

7. The device of claim 6 wherein the assessment mechanism is capable to detect at least one of the conditions selected from the group consisting of deterioration, cracking and wire recession capable of leading to structure failure.

8. The device of claim 6 wherein the assessment mechanism indicates a re-check of the railroad tie at a time interval.

9. The device of claim 6 wherein if the apparent depth is equal to or less than the nominal thickness but greater than a distance from a top surface of the railroad tie to a bottom wire line, the railroad tie is determined to be structurally sound.

10. A system for performing an in-track condition assessment of a plurality of concrete railroad ties utilizing impact echo impulses, comprising:
a track mechanism; and
a plurality of devices according to claim 1 which are mounted to the track mechanism; and
wherein the track mechanism moves in a linear direction to position each of the plurality of devices on an upper surface of an end of the railroad tie.

11. A method for performing an in-track condition assessment of a concrete railroad tie utilizing impact echo pulses, comprising:
positioning a tool on a top surface toward an end of the railroad tie, the tool comprising:
a base;
a plurality of transducers attached to the base which are capable of detecting an echo of a sound wave;
a single impinger connected to the plurality of transducers which is capable of imparting a sound wave;
imparting a sound wave from the impinger below the top surface of the railroad tie;
detecting with the transducers an echo of the sound wave;
determining a depth measurement from the echo;
comparing the depth measurement to a nominal thickness of the railroad tie; and
assessing if the railroad tie is structurally sound or unsound.

12. The method of claim 11 wherein assessing comprises identifying at least one condition selected from the group consisting of deterioration, cracking and wire recession capable of leading to structure failure.
